(19) United States
(12) Patent Application Publication
(10) Pub. No.: US 2007/0141597 A1
(43) Pub. Date: Jun. 21, 2007

(54) Biomimetic Biodetector of Toxins, Viruses, Bacteria, and Biological Factors

(76) Inventor: H. James Harmon, Edmond, OK (US)

Correspondence Address: FELLERS SNIDER BLANKENSHIP;BAILEY & TIPPENS, THE KENNEDY BUILDING, 321 SOUTH BOSTON SUITE 800, TULSA, OK, 74103-3318, US

(21) Appl. No.: 11/552,814

(22) Filed: Oct. 25, 2006

Related U.S. Application Data
(60) Provisional application No. 60/729,950, filed on Oct. 25, 2005.

(51) Int. Cl. C12Q 1/70; C12Q 1/68

(52) U.S. Cl. 435/6; 435/5

(57) ABSTRACT

A method for detecting a target agent in an environment is disclosed. The method includes preparing a biomimetic ligand, the ligand providing a site at which the target agent will bind. The method also includes covalently bonding a colorimetric indicator with the biomimetic ligand on a non active site of the ligand to produce a colorimetric capture entity, and interrogating the colorimetric indicator to determine if an analyte has bound to the colorimetric capture entity.

### CROSS REFERENCE TO RELATED APPLICATIONS

This application claims the benefit of U.S. Provisional Patent Application Ser. No. 60/729,950, filed Oct. 25, 2006, which is hereby incorporated by reference.

### STATEMENT REGARDING FEDERALLY SPONSORED RESEARCH

The Government of the United States of America has certain rights in the invention pursuant to Grant No. EEC-0503831 awarded by the National Science Foundation and funds provided by this award include support from Department of Army—U.S. Army Defense Ammunition Center.

### FIELD OF THE INVENTION

The disclosure relates to detection of toxins, viruses, bacteria, and biological factors in general and, more specifically, to detection of the same with spectrometry.

### BACKGROUND OF THE INVENTION

Functional biosensors are needed for many purposes. Military and related services require biosensors for the detection of biological weapons of mass destruction. The medical community needs biosensors for medical diagnosis and treatment. Currently medical diagnosis as well as military detection requires longer-than-desirable times as well as expensive complicated equipment and trained personnel. This is especially apparent in conventional medical laboratories where samples are streaked, plated, and grown on different species-specific media. A real-time selective and sensitive biological detector at a reasonable price with minimal operator involvement is desirable.

Biosensors (or biological detectors) are desirable to detect three (3) major biological entities: bacteria, viruses, and toxins. The properties of these three (3) classes are quite different; for example, only bacteria are “alive”. They all share one commonality, however, in that they must bind a host/target cell surface to cause their respective effects.

As described in U.S. Pat. No. 6,821,738, incorporated herein by reference, complexes of protein with colorimetric compounds may be used to detect the presence of very low concentrations of hazardous or chemical warfare agents. Changes in the spectrum of a properly chosen colorimetric compound can be used as a “real-time” indicator to detect the presence of dangerous substances such as nerve agents, organophosphates (OP), and other chemical warfare agents. The electron distribution in a colorimetric compound is altered by its immediate environment. Changes in electron distribution result in corresponding changes in the spectrum of the colorimetric indicator. Thus, an indicator for use in detecting hazardous compounds may be created by monitoring specific lights wavelengths in the spectrum of a colorimetric compound of choice. Further, because of the multiplicity of absorbance bands in various of these indicators, unique spectral “signatures” may be developed for use in subsequent detection. As one example, an AChE-based chemical biosensor is created by binding to the AChE molecule a porphyrin which inhibits it and which reversibly binds at the active site where substrate and/or nerve gasses and other inhibitors will bind. One such preferred porphyrin is TPPS.sub.1 (i.e., monosulfonate tetraphenyl porphyrin). When such a biosensor is exposed to nerve agents, etc., the OP compound will displace the selected porphyrin, thereby resulting in a change in the spectral properties of the biosensor.

Binding proteins such as AChE may not be able to capture or detect all analytes that are of interest. These may include bacteria, viruses, certain toxins, and biofactors. Moreover, it may not always be possible or desirable to incorporate a porphyrin or other colorimetric indicator into the active site of a protein such as AChE. Obviously, this is so when AChE is not used as a capture molecule but other circumstances may arise in which the active site of an AChE molecule is not an appropriate location to bind or reversibly bind a colorimetric indicator such as a porphyrin.

What is needed is a system and method for addressing the above, and related, problems as encountered in connection with the detection of toxins, viruses, bacteria, and other biological factors.

### SUMMARY OF THE INVENTION

The present invention disclosed and claim herein, in one aspect thereof, comprises a method of detecting toxins, viruses, bacteria, and other biological factors or entities. The method includes covalently binding a colorimetric indicator with a receptor molecule or a biomimetic analog thereof capable of binding the biological to create a receptor-indicator hybrid, the colorimetric indicator having a known spectrometric profile. The method also includes binding the receptor-indicator hybrid to a testing substrate, exposing the testing substrate in a test environment, performing a spectral interrogation of the receptor-indicator hybrid to determine a spectrographic profile thereof, and determining the binding of a specified biological factor based upon the change the spectrographic profile.

A better understanding of the present invention, its several aspects, and its advantages will become apparent to those skilled in the art from the following detailed description, taken in conjunction with the attached drawings, wherein there is shown and described the preferred embodiment of the invention, simply by way of illustration of the best mode contemplated for carrying out the invention.

### BRIEF DESCRIPTION OF THE DRAWINGS

FIG. 1 is a diagrammatic view of glycophorin molecules in a red blood cell (RBC).

FIG. 2a is a photomicrograph of a ConA-porphyrin surface before exposure to RBCs.

FIG. 2b is a photomicrograph of FIG. 2a after exposure to RBCs.

FIG. 3 is a graph showing a spectrum of the ConA-TPPS slide before and after exposure to RBCs.

FIG. 4 is a graph showing spectral changes caused by displacement of the TPPS from ConA.

FIG. 5 is a graph showing the spectrum of glycophorin with porphyrin substrated from the spectrum of the ConA-exposed slides.

FIGS. 6A and 6B are three-dimensional representations of a comparison of porphyrin juxtaposed with jacalin (FIG. 6a) and ConA (FIG. 6b) in the corresponding carbohydrate-binding sites.

FIG. 7 is a graph of the absorbance spectrum of ConA bound to T-antigen where ConA was immobilized as a monolayer on a glass slide and TPPS was incorporated into it.

FIG. 8A illustrates a side view of a portion of an evanescent absorbance measurement device according to aspects of the present disclosure.

FIG. 8B is a top view of the portion shown in FIG. 8A.

FIG. 8C is a schematic view of the device of FIGS. 8A-8B.

FIG. 9 illustrates the difference spectra of TPPS plus naphthalene minus TPPS for four different concentrations of naphthalene.

FIG. 10 is illustrates a simplified schematic of a detector according to aspects of the present disclosure.

FIG. 11 is illustrates a detection circuit using a fluorescent light source, according to aspects of the present disclosure.

FIG. 12 illustrates a porphyrin-receptor hybrid according to aspects of the present disclosure.

FIG. 13 illustrates the absorbance spectra of sialyllactosamine porphyrin.

FIG. 14 illustrates the absorbance spectra of sialyllactosamine porphyrin exposed to NA.

FIG. 15 illustrates the absorbance spectra of sialyllactosamine porphyrin challenged with human influenza virus.

FIG. 16 illustrates the structure of the membrane glycolipid GM₁.

FIG. 17 illustrates the absorbance spectra of immobilized GM₁ and porhpyrin exposed to Cholera Toxin B subunit.

FIG. 18 is a graph illustrating the change in absorbance spectra of GM₁ porphyrin slides exposed to Cholera Toxin B subunit.

### DESCRIPTION OF THE PREFERRED EMBODIMENTS

Before explaining the present invention in detail, it is important to understand that the invention is not limited in its application to the details of the embodiments and steps described herein. The invention is capable of other embodiments and of being practiced or carried out in a variety of ways. It is to be understood that the phraseology and terminology employed herein is for the purpose of description and not of limitation.

The basis of the sensors of the present disclosure is the binding of a biological entity to its appropriate receptor. The attack of a biological involves binding to or adhering to the target receptor cell. Bacteria, viruses, and toxins attack and involve different cells/tissues because only certain cells/tissues have the proteins/receptors/enzymes/etc. that bind to the entity. However, every bacteria, virus, and pathogen uses a receptor on the host cell surface that allows for binding or penetration, etc. Bacteria have molecules on their outer surface that bind to (are recognized) by specific receptor molecules on the host cell outer surface. Some bacteria show great specificity while others do not. Viruses typically show great specificity. Regardless, the initial step in infection is the binding of the biological to a cell surface receptor molecule. The binding of the biological to a cell surface receptor molecule is what is utilized in certain implementations of the present invention. Further, a molecule mimicking or functionally similar to a receptor (referred to herein as a biomimetic analog), can be used to bind such biologicals rather than having to use a purified receptor protein. In a one embodiment, a porphyrin indicator is covalently bound to the receptor whereby the binding of the target biological creates a spectral change in the complex. The change in spectral properties can be detected by means of absorbance or fluorescence spectrography.

The receptor for a particular biological entity (or a molecule that is known to bind the entity, as in the case of toxins) is preferably immobilized onto a glass surface. A porphyrin or other colorimetric indicator molecule is covalently bonded to the immobilized receptor as well. When the receptor-porphyrin complex binds the biological, the porphyrin is either removed from the receptor or bound in a receptor-porphyrin-biological complex. In either case, the spectrum of the porphyrin changes and this change is detected using the optical platform as described in USPN 6,821,738. Only sample need be applied to the specific surface.

Toxins may also be detected as describe above. In general terms, a binding membrane or binding molecule is immobilized and incorporated with a colorimetric molecule such as porphyrin. When the toxin binds, the spectral properties of the porphyrin is altered this indicating the presence of the toxin.

The present invention will be further understood with reference to the following non-limiting experimental examples. Tables 1-3 show some of the known receptor molecules and/or binding molecules that bind to viruses, bacteria, and toxins.

TABLE 1

| | | Heparin | | CAR; | Human | Porcine | | Galactosyl |
|---|---|---|---|---|---|---|---|---|
| Virus | Glycoporphyrins | Sulfate | CR2 (CD21) | Ig-based | NH₂peptidase N | NH₂peptidase | CD4 | ceramide |
| Herpes simp. 1 | X | | | | | | | |
| Herpes simp. 2 | | X | | | | | | |
| Epstein-Barr | | | X | | | | | |
| Cocksackie and | | | | X | | | | |
| adenovirus | | | | | | | | |
| Coronavirus | | | | | X | | | |
| and SARS | | | | | | | | |
| Porcine resp | | | | | | X | | |
| virus | | | | | | | | |
| HIV | | | | | | | X | X |
| Parvovirus | | | | | | | | |
| Sendai virus | | | | | | | | |
| Respiratory | | | | | | | | |
| syncytial virus | | | | | | | | |
| flavivirus | | | | | | | | |
| picornavirus | | | | | | | | |
| alphavirus | | | | | | | | |
| Influenza A | | | | | | | | |
| Measles | | | | | | | | |
| Duck hepatitis B | | | | | | | | |
| Mouse hapatitis | | | | | | | | |
| West nile | | | | | | | | |
| Marburg | | | | | | | | |
| Vaccinia (pox) | | | | | | | | |
| Rabies | | | | | | | | |
| Dengue | | X | | | | | | |

| | | | | | | | Sialic acid | Sialic acid α |
|---|---|---|---|---|---|---|---|---|
| Virus | Glycophorin A | GD1a | GT1b | GQ1b | Haparan sulfate | Heparin | 2-6 linkages | 2,6- or 2,3 gal |
| Herpes simp. 1 | | | | | X | | | |
| Herpes simp. 2 | | | | | X | | | |
| Epstein-Barr | | | | | | | | |
| Cocksackie and | | | | | | | | |
| adenovirus | | | | | | | | |
| Coronavirus | | | | | | | | |
| and SARS | | | | | | | | |
| Porcine resp | | | | | | | | |
| virus | | | | | | | | |
| HIV | | | | | | | | |
| Parvovirus | X | | | | | | | |
| Sendai virus | X | X | X | X | X | X | | |
| Respiratory | | | | | X | X | | |
| syncytial virus | | | | | | | | |
| flavivirus | | | | | X | | | |
| picornavirus | | | | | X | | | |
| alphavirus | | | | | X | | | |
| Influenza A | | | | | | | X | X |
| Measles | X | | | | | | | |
| Duck hepatitis B | | | | | | | | |
| Mouse hapatitis | | | | | | | | |
| West nile | | | | | | | | |
| Marburg | | | | | | | | |
| Vaccinia (pox) | | | | | X | | | |
| Rabies | | | | | | | | |
| Dengue | | | | | | | | |

| | | Glycine | Mouse | | | Nicotinic | GAGs: | Carboxy- |
|---|---|---|---|---|---|---|---|---|
| | CD46, | decar- | biliary | | Folate | Ach | chondroitin | peptidase |
| Virus | CD150 | boxylase | glycoprot | Integrins | receptor | receptor | SO₄ | D |
| Herpes simp. 1 | | | | | | | | |
| Herpes simp. 2 | | | | | | | | |
| Epstein-Barr | | | | | | | | |
| Cocksackie and | | | | | | | | |
| adenovirus | | | | | | | | |
| Coronavirus | | | | | | | | |
| and SARS | | | | | | | | |
| Porcine resp | | | | | | | | |
| virus | | | | | | | | |
| HIV | | | | | | | | |
| Parvovirus | | | | | | | | |
| Sendai virus | | | | | | | X | |
| Respiratory | | | | | | | X | |
| syncytial virus | | | | | | | | |
| flavivirus | | | | | | | | |
| picornavirus | | | | | | | | |
| alphavirus | | | | | | | | |
| Influenza A | | | | | | | | |
| Measles | X | | | | | | | |
| Duck hepatitis B | | X | | | | | | X |
| Mouse hapatitis | | | X | | | | | |
| West nile | | | | X | | | | |
| Marburg | | | | | X | | | |
| Vaccinia (pox) | | | | | | | X | |
| Rabies | | | | | | X | | |
| Dengue | | | | | | | | |

TABLE 2

| BACTERIUM | α₁, acid glycoprotein | Fetuin | Asialofetuin | Laminin | Lactoferrin | Fibronectin | Glycophorin A | 3′ sialyllactose |
|---|---|---|---|---|---|---|---|---|
| Helicobacter | X | X | X | | | | | |
| H. pylori | | X | | X | X | X | X | X |
| Lactobacillus | | | | | | X | | |
| E. coli | X | X | | X | | | X | |
| Uropathic | | | | | | | | |
| E. coli | | | | | | | | |
| Streptococcus | | X | X | X | | | | |
| pyogenes | | | | | | | | |
| Staphylococcus | | X | X | X | X | X | | |
| aureus/sanguis | | | | | | | | |
| Propionibacter | | | | | | | | |
| Yersinia | | | | | | | | |
| enterocolitica | | | | | | | | |
| Listeria | | | | | | | | |
| monocytogenes | | | | | | | | |
| Mycoplasm | X | | | | | | X | |
| pneumoniae | | | | | | | | |
| Mycobacterium | | | | | | | | |
| tuberculosis | | | | | | | | |
| Salmonella | | | | | | | | |
| typhinurium | | | | | | | | |
| Shigella | | | | | | | | |
| flexneri | | | | | | | | |
| Klebsellia | | | | | | | | |
| pneumoniae | | | | | | | | |
| Vibrio cholera | | | | | | X | | |
| Pseudomonas | | | | | | | | |
| aeruginosa | | | | | | | | |
| Gram | | | | | | | | |
| negatives | | | | | | | | |
| Pneumocystis | | | | | | | | |
| carinii | | | | | | | | |
| Streptococcus | | | | | | | | |
| pneumoniae | | | | | | | | |
| | | | | | | A1-4 | | |
| | | Lotus | Haparan | | NeuAc | galabiose | | |
| BACTERIUM | Collagen | agglutinin | sulfate | Heparin | α-2,3 res. | ends | GB3Cer, GB4Cer | Thyroglobulin |
| Helicobacter | | | | | | | | |
| H. pylori | X | X | X | X | | | | |
| Lactobacillus | X | | | | | | | |
| E. coli | | | | | X | | | |
| Uropathic | | | | | | X | X | |
| E. coli | | | | | | | | |
| Streptococcus | | | X | | | | | X |
| pyogenes | | | | | | | | |
| Staphylococcus | X | | X | X | | | | |
| aureus/sanguis | | | | | | | | |
| Propionibacter | | | | | | | | |
| Yersinia | | | X | X | | | | |
| enterocolitica | | | | | | | | |
| Listeria | | | X | X | | | | |
| monocytogenes | | | | | | | | |
| Mycoplasm | | | | | | | | |
| pneumoniae | | | | | | | | |
| Mycobacterium | | | | X | | | | |
| tuberculosis | | | | | | | | |
| Salmonella | | | | | | | | |
| typhinurium | | | | | | | | |
| Shigella | | | | | | | | |
| flexneri | | | | | | | | |
| Klebsellia | | | | | | | | |
| pneumoniae | | | | | | | | |
| Vibrio cholera | | | | | | | | |
| Pseudomonas | | | | | | | | |
| aeruginosa | | | | | | | | |
| Gram | | | | | | | | |
| negatives | | | | | | | | |
| Pneumocystis | | | | | | | | |
| carinii | | | | | | | | |
| Streptococcus | | | | | | | | |
| pneumoniae | | | | | | | | |

| | | Lactoneo- | | Chondroitin | Serum | Hemo- | Fibri- | Vitro- |
|---|---|---|---|---|---|---|---|---|
| BACTERIUM | Mucin | tetralose | AsialoGM1,2 | sulfate | Amyloid A | globin | nogen | nectin |
| Helicobacter | | | | | | | | |
| H. pylori | | | | | | | | X |
| Lactobacillus | | | | | | | | |
| E. coli | | | | | X | | | X |
| Uropathic | | | X | | | | | |
| E. coli | | | | | | | | |
| Streptococcus | X | | | | | | | |
| pyogenes | | | | | | | | |
| Staphylococcus | | X | | | | X | X | X |
| aureus/sanguis | | | | | | | | |
| Propionibacter | | | X | | | | | |
| Yersinia | | | | | | | | |
| enterocolitica | | | | | | | | |
| Listeria | | | | | | | | |
| monocytogenes | | | | | | | | |
| Mycoplasm | | | | X | | | | |
| pneumoniae | | | | | | | | |
| Mycobacterium | | | | | X | | | |
| tuberculosis | | | | | | | | |
| Salmonella | | | | | X | | | |
| typhinurium | | | | | | | | |
| Shigella | | | | | X | | | |
| flexneri | | | | | | | | |
| Klebsellia | | | | | X | | | |
| pneumoniae | | | | | | | | |
| Vibrio cholera | | | | | X | | | |
| Pseudomonas | | | | | X | | | |
| aeruginosa | | | | | | | | |
| Gram | | | | | X | | | |
| negatives | | | | | | | | |
| Pneumocystis | | | | | | | | X |
| carinii | | | | | | | | |
| Streptococcus | | | | | | | | X |
| pneumoniae | | | | | | | | |

TABLE 3

| | | | Acid-solubilized | 1 −> 4 β | | Gb3 | | α 1,4 | Glycophorin | |
|---|---|---|---|---|---|---|---|---|---|---|
| TOXINS | Chitotriose | Chitotetraose | chitin | linked NAg | GlcNAc | globotriasylcer | G_{M1} | galabiose | A or B | GD1a |
| Shigella | X | X | X | X | X | X | X | X | | |
| Pertussis | | | | | | | | | | |
| Tetanus | | | | | | | | | | X |
| Botulism | | | | | | | | | | X |
| Diphtheria | | | | | | | | | | |
| Cholera | | | | | | | X | | X | |
| El Tor | | | | | | | | | | |
| hemolysin | | | | | | | | | | |
| E. coli Heat- | | | | | | | X | | | |
| labile toxin | | | | | | | | | | |
| Alpha | | | | | | | | | X | |
| hemolysin | | | | | | | | | | |
| aerolysin | | | | | | | | | X | |

| | | | | | | | | NAG, | | |
|---|---|---|---|---|---|---|---|---|---|---|
| | | | | | | | | mannose, | | |
| TOXINS | GT1b | GQ1b | Fetuin | Galactomacropeptide | Lactoferrin | ATP | heptapeptide | α-mann | glycoproteins | Glycophorin B |
| Shigella | | | | | | | | | | |
| Pertussis | | | X | | | | X | | | |
| Tetanus | X | X | | | | | | | | |
| Botulism | X | X | | | | | | | | |
| Diphtheria | | | | | | X | | X | X | |
| Cholera | | | X | X | ?? | | | | | |
| El Tor | | | | | | | | | | X |
| hemolysin | | | | | | | | | | |
| E. coli Heat- | | | | | | | | | | |
| labile toxin | | | | | | | | | | |
| Alpha | | | | | | | | | | |
| hemolysin | | | | | | | | | | |
| aerolysin | | | | | | | | | | |

One example of the present method may be illustrated using Table 1. Commercially available glycophorin, a protein found on the surface of red blood cell membranes, can be immobilized and have a porphyrin incorporated into it. According to Table 1, parvovirus, Sendai virus, and measles virus will bind thereto and we expect a change in the spectrum of the porphyrin bound to glycophorin. This would mean that the sample contains one (1) or more of those three (3) viruses. This single step has dramatically narrowed the possible identity of the potential biological in the sample.

If the sample is placed onto a slide of immobilized heparan-porphyrin complex and a spectral change is observed, this can only be due to the presence of Sendai virus since that is the only entity of the three (3) that binds to heparan sulfate. Similarly, measles is the only one of the three (3) that binds to CD46. If the sample does not cause a spectral change in either CD46 or heparan sulfate-coated slides but causes a change in the spectrum of glycophorin-porphyrin coated slides, the sample contains parvovirus.

In some cases, a single slide may not identify an agent. However, the use of logical tests (all require only a few seconds and only a drop of sample) can determine what biologicals are present in the sample. Further, the intensity of the spectral change indicates the number of biological entities present.

The examples described below illustrate viable embodiments of the present disclosure including binding of cells, binding of a receptor to a simulated “toxin”/binding of the simulated “toxin” to the receptor, and binding of a specific antigen diagnostic of cancer to a receptor.

### Binding of Cells

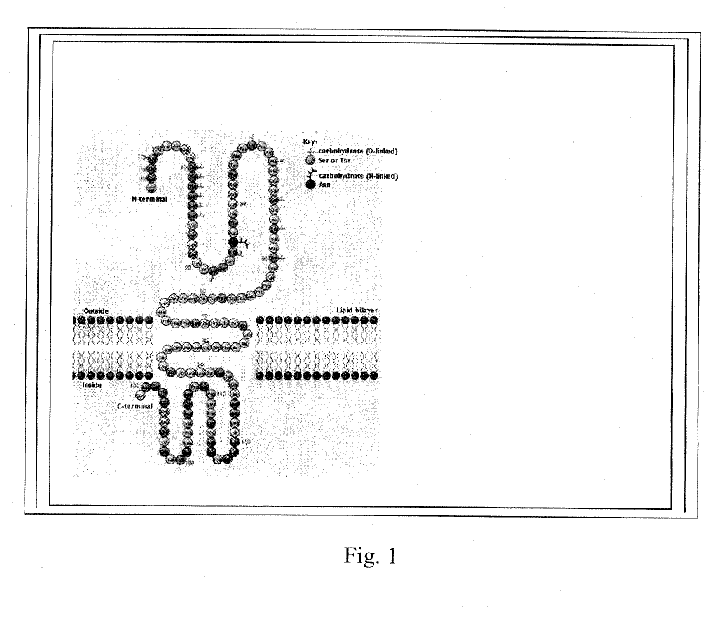

Referring now to FIG. 1, a diagrammatic view of glycophorin molecules in a red blood cell (RBC) is shown. Red blood cells from humans contain an incredible 500,000 to 1 million glycophorin molecules exposed on each cell's surface. Glycophorin is a carbohydrate-containing protein that binds to a broad class of molecules called lectins (e.g., ricin toxin is a lectin). One of those lectins is called conconavalin A (ConA).

In this experiment, ConA was covalently immobilized onto a glass slide and the porphyrin tetraphenylporphyrin sulfonate (TPPS) was added to the surface and allowed to react (bind) to the ConA. The excess was washed off and the spectrum of the ConA-porphyrin complex measured. Red blood cells (RBCs) were added to the slide, allowed to react, and then washed off. The slide was then examined for bound RBCs and the spectrum of the slide after exposure to RBCs was measured and compared to the pre-RBC exposure surface.

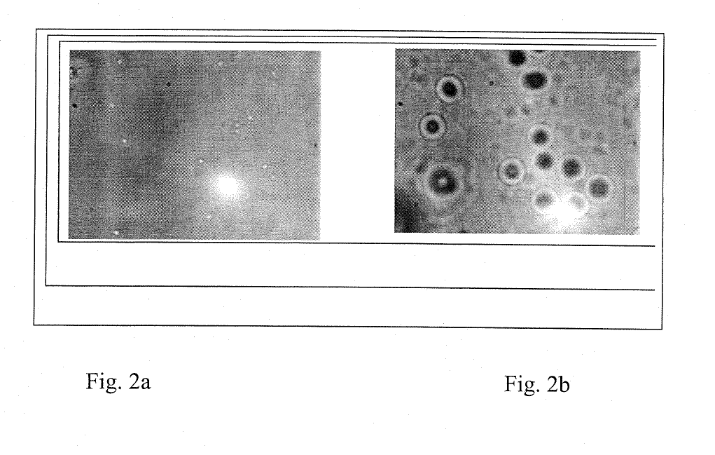

Referring now to FIG. 2A, a photomicrograph of a ConA-porphyrin surface before exposure to RBCs is shown. Referring also to FIG. 2B, a photomicrograph of FIG. 2A after exposure to RBCs is shown. The left slide (FIG. 2A) is the ConA-porphyrin surface (with dirty optics) before exposure to RBCs while the photomicrograph on the right (FIG. 2B) is the same slide after exposure to RBCs. The red blood cells are immobilized on the slide surface and easily seen.

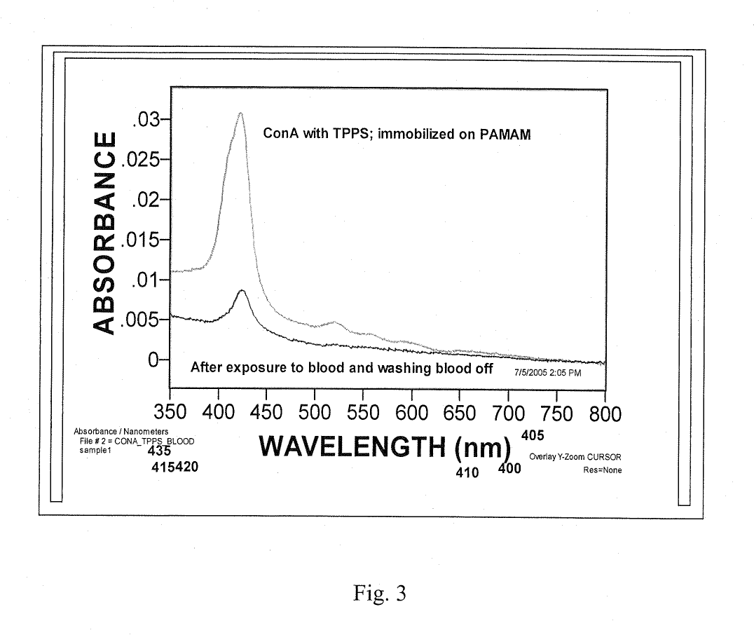

Referring now to FIG. 3, a graph showing a spectrum of the ConA-TPPS slide before and after exposure to RBCs is shown. The RBCs displaced the TPPS from the ConA, resulting in a marked decrease in the intensity of the absorbance of blue light (413 nm) by TPPS. From this it may be observed that cells can be bound (and concentrated) onto a specific receptor surface and this can be detected spectrally as well as with a microscope.

### Binding of a Receptor and Its Simulated Toxin

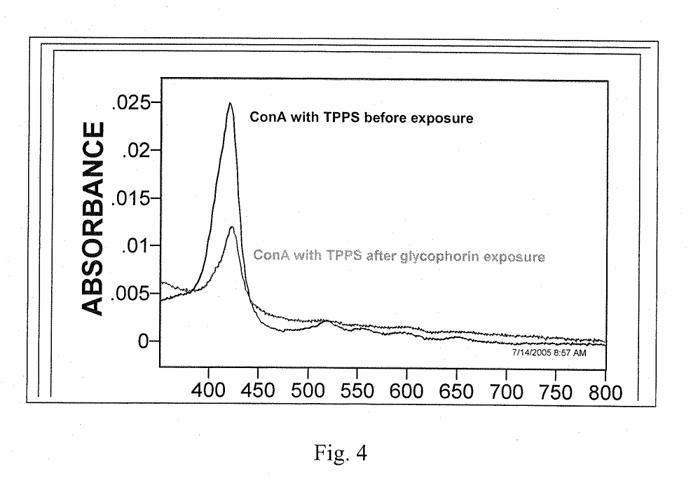

Referring now to FIG. 4, a graph showing spectral changes caused by displacement of the TPPS from ConA is shown. ConA on the slide also binds isolated glycophorin in solution. The glycophorin molecules are too small to be seen with a microscope, but the spectral changes caused by displacement of the TPPS from ConA are seen as a decrease in the absorbance intensity of the bound TPPS, as shown in FIG. 4.

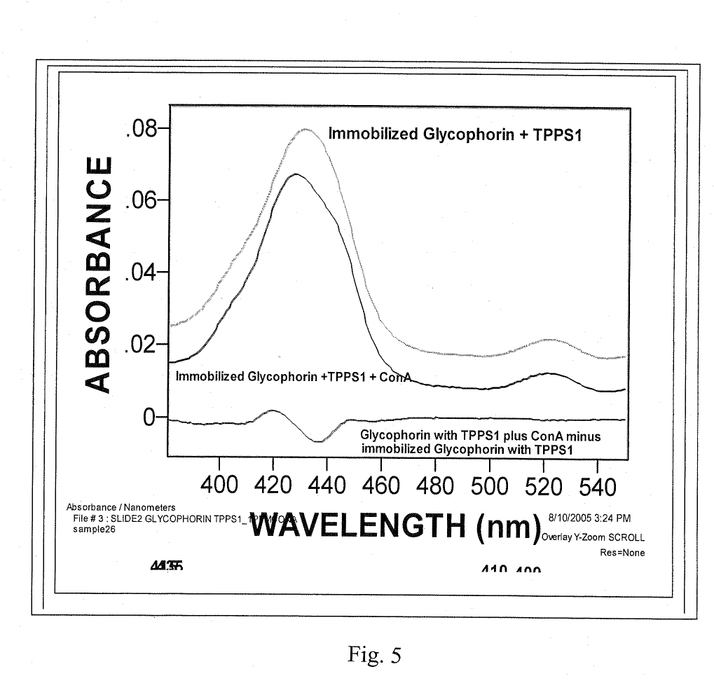

The converse is also true. When a toxin binds to its immobilized receptor, the spectrum of a porphyrin bound to the receptor will change whether the porphyrin is displaced or not. Referring now to FIG. 5, a graph showing the spectrum of glycophorin with porphyrin substrate from the spectrum of the ConA-exposed slides is shown. Here, we let glycophorin act as the simulated membrane “receptor” (it is in the membrane and is isolated) that will bind to a simulated “toxin”/biological, ConA. Glycophorin was immobilized on the slide and another porphyrin was incorporated into the glycophorin. Exposure of the immobilized “receptor”/glycophorin-porphyrin slide to ConA (the stimulant biological) results in spectral changes in the porphyrin. Here, the porphyrin is not displaced; it appears that the porphyrin is “sandwiched” between the glycophorin and ConA, resulting in a change in the spectrum (a loss of absorbance intensity is also a change). The “difference” spectrum of the spectrum of glycophorin with porphyrin subtracted from the spectrum of the ConA-exposed slide is also shown. The loss of absorbance at 436 nm (trough) indicates that the porphyrin has complexed with the ConA to form a porphyrin complex that absorbs at 424 nm.

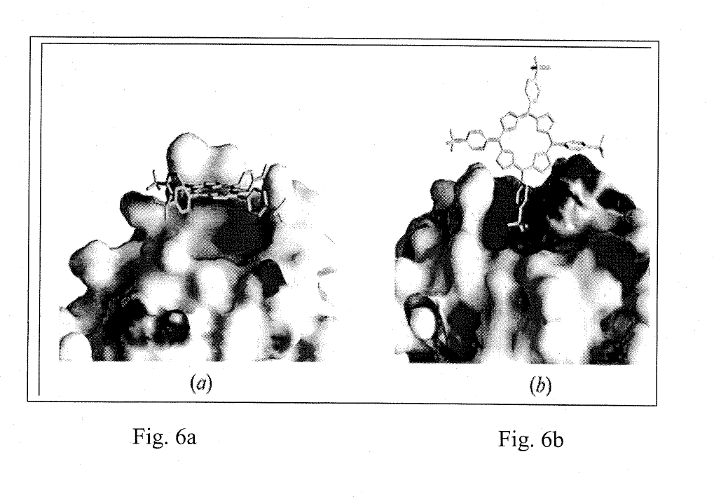

Referring now to FIGS. 6A and 6B, three-dimensional representations of a comparison of porphyrin juxtaposed with jacalin (FIG. 6A) and ConA (FIG. 6B) in the corresponding carbohydrate-binding sites. Porphyrin is shown in sticks and jacalin and ConA, incorporating the corresponding sandwiched water molecules, are molecular-surface representations. These FIGS are derived from Goel, M. et al Acta Cryst. D 60, 281-288 (2004), which is hereby incorporated by reference. These FIGS. 6A and 6B illustrate the fact that lectins like ConA bind porphyrin in the carbohydrate-binding site as shown.

### Binding of a Specific Antigen Diagnostic of Cancer

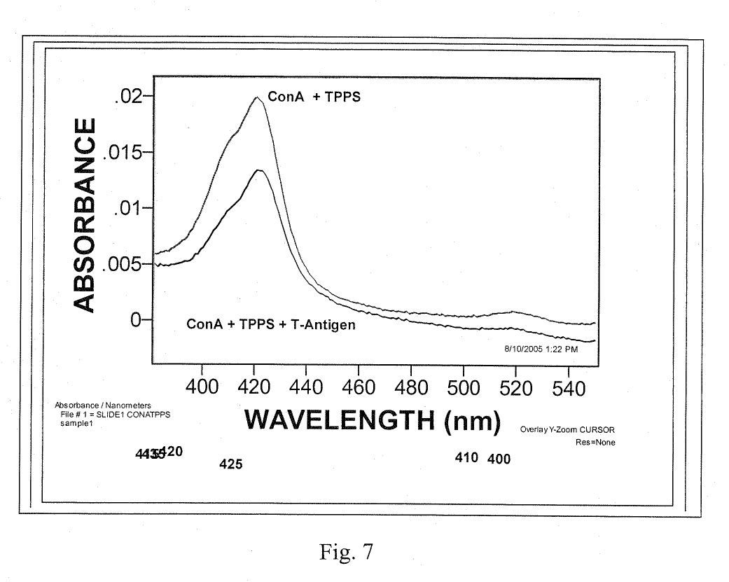

T-antigen is a disaccharide (Galβ1-3-GalNAc; galactose-N-acetylgalactose) expressed in more than 85% of human carcinomas such as colon, breast, bladder, buccal cavity, and prostate as well as on poorly differentiated cells. “Therefore, proteins which specifically bind T-antigen have potential diagnostic value” (Jeyaprakash et al J. Mol. Biol. (2002) 321, 637-645). The lectin jacalin from jack fruit (see diagram above) binds T-antigen. Other lectins bind it as well and can also be used. T-antigen is commercially available as is jacalin. Referring now to FIG. 7, a graph showing ConA bound to T-antigen where ConA was immobilized as a monolayer on a glass slide and TPPS was incorporated into it is shown. The graph further shows the effect of T-antigen challenge on the spectrum.

### Binding of a Virus

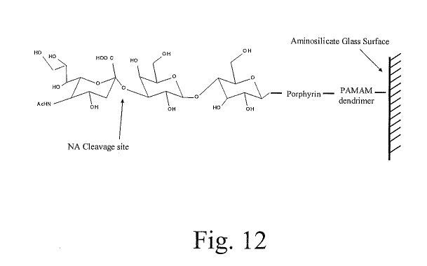

It is apparent to those familiar with the art that the previous examples of detecting biologicals involves the incorporation of a colorimetric porphyrin into/onto the target receptor (jacalin or ConA for example). However, many receptors do not have a binding or active site such as is found in enzymes or lectins into which a porphyrin can be incorporated. For example, cholera toxin binds to the receptor GM₁ which is found on the host cell surface; influenza virus binds to sialic acid compounds such as siallylactose (FIG. 12). These receptors (often lipids or carbohydrates) do not readily bind a colorimetric compound such as porphyrin. However, we can covalently bind these receptors to the colorimetric porphyrin indicator to make a porphyrin-receptor hybrid compound which acts as the appropriate receptor for the target biological and has the colorimetric indicator covalently bound; the receptor is also the indicator instead of having the indicator merely associated with the indicator. This has the advantage of the indicator being permanently bound to the receptor. The porphyrin-receptor molecule can then be covalently bound to the detecting surface. In the prior patent, U.S. Pat. No. 6,821,738 B2, the receptor protein (such as AChE) was bound to the slide and then porphyrin was added to incorporate it into the active site of the protein/enzyme. In the present embodiment, the porphyrin indicator is an integral part of the receptor (which need not be protein) with the indicator covalently bound to the receptor.

Consider the following example. The surface of Influenza virus is studded with neuraminidase (NA) and hemagglutinin (HA) proteins; both proteins bind to sialic acid with NA cleaving the sialic acid portion from the receptor. In this example, a porphyrin (tetra-aminophenyl porphyrin) is covalently bound to a glass slide coated with aminosilane and PAMAM. Siallylactose amine (SLA) which will be bound by both NA and HA of the virus coat exterior is then covalently bound to the porphyrin to make a surface depicted in FIG. 12. Binding of HA to the immobilized SLA causes a change in the spectrum of the SLA-porphyrin complex; Cleavage of the sialic acid portion from the immobilized SLA-porphyrin complex also changes the electron distribution (and hence the absorbance spectrum) of the attached porphyrin. After NA binds, however, the sialic acid portion is no longer present and the NA can leave the molecule. Thus, HA binds to the sialic acid portion resulting is a HA-SLA-porphyrin complex bound to glass; addition of NA to the SLA-porphyrin complex results if the formation of asialolactose amine-porphyrin left bound to the glass surface. Binding of HA to the surface results in the flu virus (or portion of the flu virus) being bound to the slide. Thus, the choice of appropriate receptor materials can yield an “archived” surface that contains the target biological (which can be subsequently studied, measured, or identified at a later time by other techniques, if so desired.

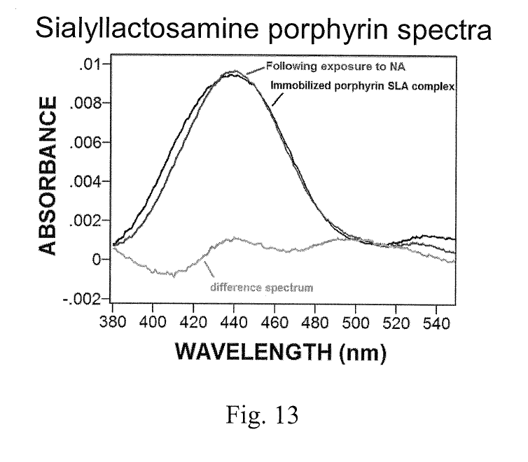

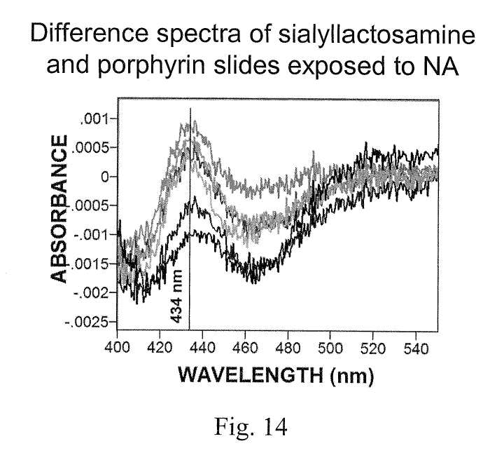

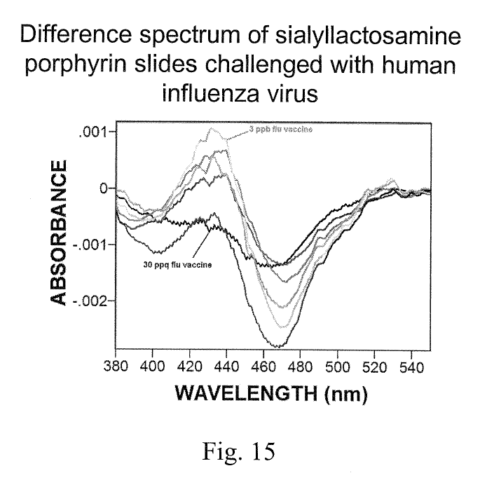

Use of the immobilized SLA-porphyin results in data in FIGS. 13-15. In FIG. 13, the spectrum of the porphyrin covalently bound to the slide and the SLA is shown before and after exposure to purified NA; difference spectra of the post minus pre-exposure surface are shown in FIG. 14 and illustrate that the porphyrin-SLA compound minus the asialo-porphyrin spectrum results in an absorbance increase at 434 nm and a decrease at approx 465 nm. The absorbance difference between the 434 nm peak and the 564 nm trough is proportional to the amount of NA added to the slide surface. If the experiment is repeated in the presence of human influenza virus, we see similar concentration-dependent difference spectra in FIG. 15 because the NA of the virus coat has acted in the same manner as isolated NA to cleave the sialic acid moiety, leaving immobilized asialolactose-porphyrin. Levels of NA down to approximately 50 ppq (0.05 parts per trillion; 5×10⁻⁵ parts per million) in solution can be detected.

It will be apparent that this detector, like the cholera toxin detecting surface involves the natural receptor which is modified by the covalent addition of the porphyrin colorimetric indicator to yield a substrate to which the biological entity will bind and, as a result of the binding, cause a change in the spectrum of the receptor molecule. The receptor IS the colorimetric indicator without the addition of other indicator molecules such as dyes, labeled probes/antibodies, etc.

### Detection of a Toxin

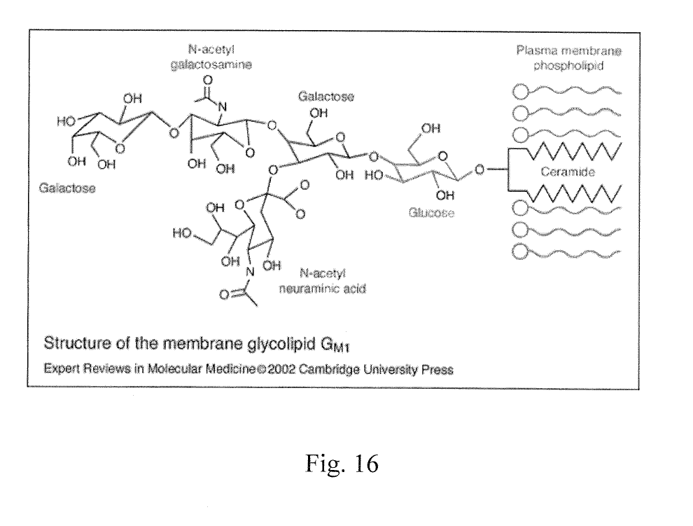

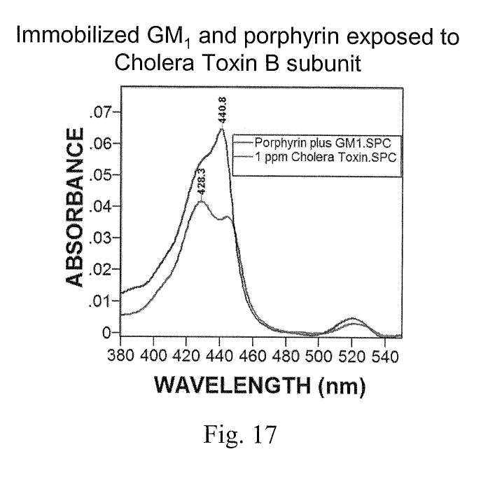

Choler toxin binds to the cell surface of the host victim at a compound called GM₁, a pentasaccharide receptor molecule shown in FIG. 16. A tetraaminophenyl porphyrin can be covalently bound to either N-acetyl group shown by a glutaraldehyde-generated Schiff's base reaction. The porphyrin can then be bound to the PAMAM bound to the glass surface; this yields a porphyrin-GM₁ hybrid immobilized to the glass slide. When cholera toxin binds to the porphyrin-GM₁ hybrid complex, the electron distribution is altered and the spectrum of the covalently bound porphyrin changes as shown in FIG. 17.

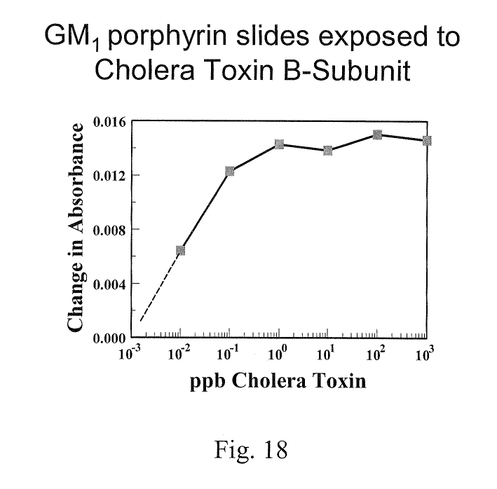

As can be seen in FIG. 17, the presence of cholera toxin decreases the absorbance at both 428 and 441 nm, but the 441 nm absorbance decreases to a far greater degree. A small shift in absorbance in the 520 nm region is also observed. Again, as with other analyte detections, the absorbance change is dependent on the amount of toxin added. As seen in FIG. 18, we have detected down to 10 parts per trillion (0.00001 parts per million) cholera toxin.

### Colorimetric Indicators

Highly colored heterocyclic compounds such as porphyrins or related compounds such as phthalocyanines may be employed as colorimetric indicators as described in U.S. Pat. No. 6,821,738. One preferred class of molecule whose optical characteristics are altered by the presence of other molecules is the porphyrins. The spectra of porphyrins is altered by the presence of numerous toxins, viruses, bacteria, and biological factors. Thus, porphyrins or similar molecules may be used as indicators for these substances for methods and devices of the present disclosure.

One preferred class of molecule whose optical characteristics are altered by the presence of other molecules is the porphyrins. The spectra of porphyrins is altered by the presence of numerous toxins, viruses, bacteria, and biological factors. Thus, porphyrins or similar molecules may be used as indicators for these substances for methods and devices of the present disclosure.

Porphyrins are nitrogen-containing compounds that are derived from the parent molecule tetrapyrroleporphin and which are very useful for purposes of the instant invention. They are classified on the basis of the nature of the side chains replacing the hydrogens at positions 1-8; methyl, ethyl, vinyl, and propionic acid are common substituents. One explanation for the observed spectral changes in the porphyrins is the alteration of pi-electrons of the porphyrins (this gives them their intense color) by the analyte molecules.

Complexation of porphyrins with metal alters the absorbance spectrum of the porphyrins. It is of interest to note that the wavelength is dependent on the metal as well as the solvent used. In a given solvent, the wavelength maxima of the different metal complexes are sufficiently different to allow spectrophotometric resolution of the different metal complexes. The absorbance spectrum and the extinction coefficient (absorptivity) of a metallo-/porphyrins are known to be affected by the solvent. The basis for these solvent-induced spectral changes is similar to the basis of the change in the wavelengths absorbed by altering the groups substituting at positions 1-8 on the porphyrin ring. Factors which cause an increase in pi-electron orbitals at the periphery of the porphyrin tend to cause red shifts of the absorbance and fluorescence (if present) bands. Red shifts are found to arise as a function of the electron affinity of side chain substituents at positions 1-8. As the electronegativity is increased, the stability of the metal chelate decreases and absorption/emission bands shift accordingly. As pi electrons are withdrawn from the periphery, the spectrum blue shifts to shorter wavelengths.

Just as the energy transitions for absorbance of photon energy are altered, so are the energy transitions involved in photon emission of absorbed energy. Thus the fluorescence spectra of porphyrins is altered, each having its unique spectrum.

In order to function as a detector for purposes of the instant invention, the detector molecule (e.g., colorimetric indicator) should be able to interact with the target analyte(s) and its spectrum (absorbance, fluorescence, or reflectance) must be altered by the interaction. Further, in order for porphyrins to catalyze an organic reaction, the porphyrin must bind, “dock” with, or somehow interact physically with the organic at least once during the catalytic process (a collisional encounter between substrate and catalyst). In those cases where a reactant [such as OH⁻, H⁺, electrons, O₂⁻, ¹Δ_{g}O₂ (singlet oxygen)] may be generated at the porphyrin and diffuse to the organic molecule, the diffusion distance must be very small (the lifetime of singlet oxygen in H₂O being on the order of nanoseconds), indicating that the porphyrin and organic are very close together, probably “docked”. Covalently binding the indicator to the receptor ensures that the porphyrin will be appropriately bound near the receptor “active” region to allow for electron redistribution to occur and the spectrum to be altered. The porphyrin now becomes an integral part of the receptor; if the receptor were viewed as a substrate to be bound to the analyte (as say in an enzyme) the porphyrin is now the substrate.

Docking of the organic, analogous to the formation of an enzyme-substrate complex, should result in a distortion of the electron distribution of both molecules. Since the pi-electron distribution of the porphyrins is responsible for their intense visible light absorbance, alterations of porphyrin spectra upon organic ligation should be seen. This idea is consistent with the changes in ε and λₘₐₓ of the Soret (400-450 nm) band (B-band) of the porphyrins by alteration of the porphyrin side chain constituents of the porphyrin ring and alteration of the spectral properties due to solvent polarity and hence differences in the electron distribution around the porphyrin plane.

Alteration of the spectrophotometric characteristics of porphyrins has been reported by, for example, D. Mauzerall (Biochem. 4, 1801-1810, 1965) and J. A. Shelnutt (J. of Phys. Chem. 87, 605-616, 1983), the disclosures of which are incorporated herein by reference. In these studies aromatic heterocyclic compounds such as phenanthrolines were complexed with porphyrin molecules; changes in pi-orbital density were observed, leading to changes in visible light absorbance, fluorescence, and Raman spectra.

Close interaction of porphyrins and organics resulting in the quenching of porphyrin absorbance and fluorescence spectra have been reported. Further, porphyrins have been used in the shape-selective separation of aromatics and are particularly useful in the separation of fullerenes. Soret λₘₐₓ positions have been observed to change in the presence of organic polycyclics, the shift in λₘₐₓ being proportional to the energy of association with the porphyrin. This suggests that the stronger the interaction between organic and porphyrin, the greater the shift in wavelength.

The use of porphyrins and phthalocyanines as sensor indicators is gaining in popularity. Wavelength shifts as colorimetric indicators have been used to sense the presence of pentachlorophenol, cysteine and histidine, and quinones. The binding of the quinone has been determined to be via multiple H-bonds between the quinone and the OH-naphthyl subgroups of the porphyrin as well as between the quinone and the COO⁻ groups of Zn-porphyrin dinitrobenzoic acid. A gas sensor which measures CO, NO₂, and H₂S has been designed using metalloporphrin Langmuir-Blodgett films deposited on a field-effect transistor. Dziri, L., Bousaad, S., Tao, N. and, Leblanc, R. M. (1998) Langmuir 14, 4853-4859.

In related work, the photo-induced energy transfer between two porphyrins which have been co-deposited as solid film on TiO₂ has been measured, indicating the ability of energy transfer between porphyrins and their near (docked) neighbor. Illumination of a donor molecule elicits energy changes in the acceptor porphyrin although no electron transfer occurred, indicating that changes in one molecule elicit spectral changes in another neighboring acceptor molecule. This is similar to the recognition of specific DNA sequences using oligonucleotide-derivatized polypyrroles by voltammetry and the interaction of porphyrin-thymidine complex with DNA in which the H-bonding of the porphyryl-thyrnidine with adenine results in an 8 nm red shift in the porphyrin Soret band. Thus, alteration in the electron density due to interaction and/or binding with another molecule, even at the periphery of then porphyrin, results in spectroscopic changes.

Turning now to a discussion of possible embodiment of a device based upon the methods disclosed herein, in one embodiment, as a first step, microscope slides are obtained that have been coated with amino groups. Slides sold under the trademark “Probe-On” from the company “Fisher Scientific” are particularly suitable for this purpose. Alternatively, a plain glass slide might be prepared by treating it with a chemical such as 3-aminopropyltriethoxy silane, which would “functionalize” its surfaces and result in free amino groups that are bound thereto. It should be noted that a glass slide is only one possible choice and plastic, silica, quartz, plexiglass, or many other materials might be used instead. For ease in later evaluating the detector, the material on which the amino groups are found will be substantially optically transparent.

Given a coated microscope slide as described previously, the amino groups present thereon are then preferably reacted with glutaraldehyde to form a Schiff's base, which can then be used to bind a preferred dendrimer which is sold under the trademark “Starburst” by Aldrich Chemical. The trademarked dendrimer is preferred because it has 64 amino termini, which effectively amplifies the number of binding sites by a factor of 64. The 64 amino sites are then reacted with glutaraldehyde to form a Schiff's base and then the desired capture molecule or entity is bound. Therefore, the net result is a layer of enzyme that is covalently bound to the glass, in effect, a “sandwich” of glass/Starburst/capture molecule. As a next step the porphyrin may be attached to the receptor molecule by several means of covalent linkages including but not limited to generating a Schiff's base between the molecules (if they contain amino groups) as was done for affixing the Starburst molecule to the glass slide.

Particularly for example, SLA, the receptor for influenza virus, was immobilized on ProbeOn™ Plus microscope slides to yield the surface schematically diagramed in FIG. 12. Each step was terminated by rinsing the slides with 25 mM sodium phosphate with 2.5 M NaCl. Briefly, slides were activated with 0.17 M glutaraldehyde, and exposed to PAMAM; non-bound active sites where then blocked with 1M TRIS. These steps were repeated a second time; a third application of glutaraldehyde was then applied, rinsed and the slide was then allowed to interact with a mixture of SLA, glutaraldehyde, and NH2-tetraphenyl porphyrin for 2 h. The slide was rinsed with 50 mM pH 7 sodium phosphate buffer, exposed to 1 M Tris, and rinsed again with sodium phosphate buffer. The slide surface as diagrammed in FIG. 12 is now ready for use.

For purposes of detection, the spectrum of the capture molecule/porphyrin complex, otherwise also referred to as a capture entity or capture complex, will preferably have been determined in advance of its exposure to an unknown, especially between 400 nm and 450 nm wavelengths. These spectral values can be used as baseline values and compared with the spectral values after/during exposure to check for target compounds or analytes. Later, when an inhibitor binds or interacts with the complex on the detector, it will displace or otherwise alter the spectrographic profile of the bound porphyrin. Evidence of this interaction/binding will be manifest as an increase in one spectral band will and a decrease in another band. For example, porphyrin bound to AChE absorbs at 446 nm while porphyrin free (unbound) absorbs at 429 nm. Displacement of the porphyrin from the active site of AChE will result in a decrease in absorbance at 446 nm and an increase in absorbance at 429 nm. Those of ordinary skill in the art will recognize that the specificity of the detection method suggested herein is twofold. First, only those molecules that are specifically adapted to the capture molecule with bind and displace or alter the porphyrin. And, second, the spectral wavelengths that are monitored are specific for the particular porphyrin used.

Although the detector described above could be read via conventional reflectance or absorption spectroscopy, because of the low concentration of the reagents thereon, alternative methods of assessment may be utilized. Thus, and according to another aspect of the instant disclosure, there is provided a method of reading the above-described detector via spectroscopy which is performed by “injecting” light into the slide upon which the capture molecule is bound through one of its edges, thereby creating evanescent waves which are then utilized to read the detector.

By way of general background, it is well known to those skilled in the art, that when light is incident on a medium at an angle of incidence that is greater than the critical angle, Snell's law suggests that all of the light will be reflected internally at that interface, i.e., total internal reflection will occur. However, Fresnel's equations (in concert with Maxwell's equations) predict—and, in fact, it is observed in practice—that evanescent waves will be generated at the point of total reflection. The energy of this type of wave penetrates beyond the surface of the reflecting medium and returns to its original medium unless a second medium is introduced into the region of penetration of the evanescent wave. In other words, if another medium is brought near enough to the point where total internal reflection occurs, energy in the form of evanescent waves of the same frequency as the incident light will be transmitted to the alternative medium.

Of course, if molecules are brought into proximity with a surface in which evanescent waves are propagating, the molecules will interact with those waves and attenuate them to the extent that these molecules would absorb the same wavelength in conventional light, i.e., in absorption spectroscopy. Spectroscopy via evanescent waves is a well known method of assessing the composition of low concentration materials, such as those which would be present in the preferred embodiment of the instant invention.

The evanescent waves typically extend about ¼ wavelength (e.g., 100-200 nm depending on the wavelength of the incident light) into the surrounding medium. The evanescent light waves penetrate the detector layer on the surface of the slide and interact with the chemicals present thereon. As described previously, the intensity of light that has interacted with the detecting layer is then examined at least two different spectral wavelengths for evidence of the presence of a compound (e.g., at 429 nm and at 446 nm as used in an example earlier). Preferably, the light that is supplied to the preferred slide embodiment, and which is the source of the evanescent waves, can be delivered to the detector by way of optical fibers according to methods well known to those of ordinary skill in the art.

Finally, as has been discussed previously, in the one, embodiment the instant detector will be created on glass or similar material that is optically transparent. In light of the foregoing, it should now be clear why this arrangement is preferred, as it provides a convenient way to generate and use evanescent waves when detector materials are read. Of course, preferably the glass, Plexiglas, etc., will be of a density, thickness, and configuration to encourage generation of such waves. For example, although thin planar pieces of transparent material such as microscope slides are preferred, sections of fiber optic cable which have been treated as described previously could alternatively be used.

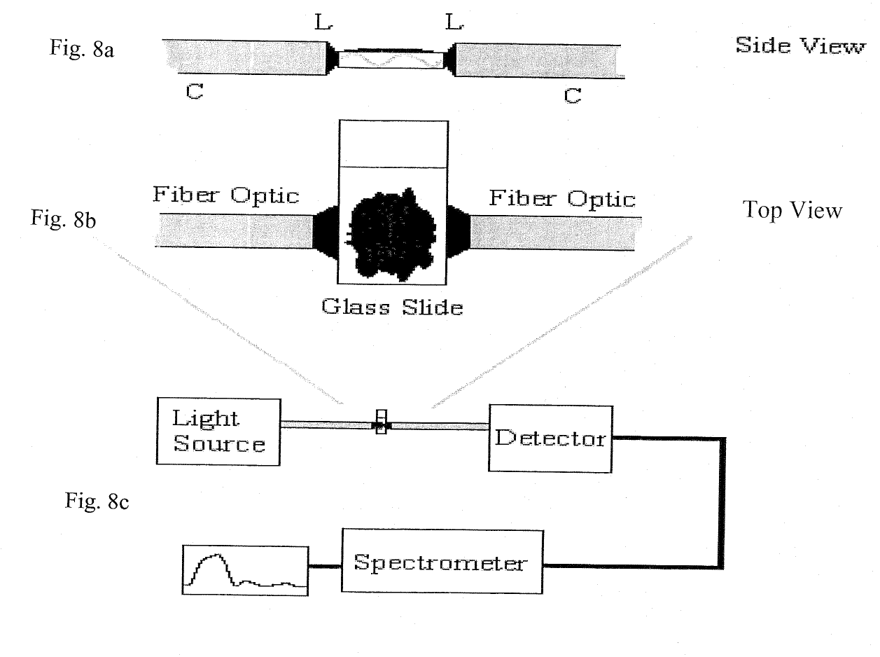

Referring now to FIG. FIG. 8A, a side view of a portion of an evanescent absorbance measurement device according to aspects of the present disclosure is shown. FIG. 8B is a top view of the portion shown in FIG. 8A. FIG. 8C is a schematic view of the device of FIGS. 8A-8B. It can be seen from these FIGS. that fiber optics may be used to illuminate the prepared slide with the output being read by a detector and/or analyzed by a spectrometer. This represents only one possible physical embodiment for utilizing the principles discussed above. As those of skill in the art will realize, additional embodiments are possible.

### The Difference Spectrum

Consider the following simple illustration. If it is assumed that an analyte shifts the λₘₐₓ of a porphyrin from 413 to 429 nm, for example, the quantitation of the analyte from the absolute spectrum of the porphyrin and analyte will require that at least 10-20% of the porphyrin be complexed. So, if 5 nmoles of porphyrin are present on an active detector surface, then 1 nmole of analyte must bind before the effect can be directly seen in the spectrum of the porphyrin. Further, the smaller the wavelength shift, the less sensitive the quantitation and the more analyte complex must be present to be measured. For example, the spectral shifts seen in FIG. 5. or FIG. 13 may be difficult to measure. Thus, detection of low concentration compounds poses a significant challenge to analytical methods that are based on direct measurement and observation of the spectrum.

Hence, the instant inventor has determined that it is preferable to detect the presence of analyte in the spectrum of the porphyrin by using a mathematical operation on the spectrum to make clearer the change in that takes place when an analyte is introduced into the system of the instant invention. That is, a central precept of the instant invention is that the preferred method of identifying low concentration compounds is to “continuously” monitor the detector compound and to continuously compare the current spectrum with a spectrum collected at some previous time. By comparing the spectrum at two different points in time, a self-calibrating and correcting procedure is developed that is much more sensitive than other approaches considered heretofore.

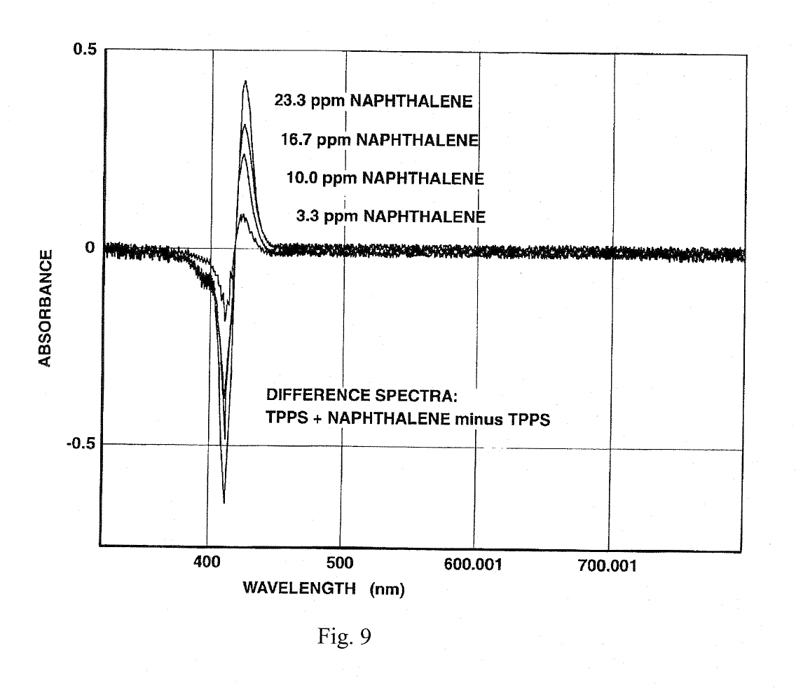

According to one embodiment, the instant method is made more sensitive to low levels of analyte by utilizing a difference spectrum, where the spectrum of unreacted porphyrin (or other colorimetric compound) is subtracted from its spectrum following exposure to the analyte. As shown in FIG. 9, the difference spectrum—which is computed by subtracting the spectrum of TPPS alone from the spectrum of TPPS+naphthalene—resembles a “1st derivative” function. The absorbance at 413 nm decreases due to loss of TPPS when naphthalene binds and shifts the wavelength (new peak) to 426 nm. Knowing the extinction coefficient of TPPS at 413 nm allows the determination of how much TPPS is lost; this is the same amount of TPPS-naphthalene complex formed.

The presence of low levels of analyte is virtually undetectable except by monitoring the change in the spectral characteristics of the detector. Unlike absolute spectra where the shape and peak wavelength of the spectral curve changes with increasing binding of analyte, the λₘₐₓ and λₘₐₓ do not change; only the intensity of the absorbance changes with changes in analyte concentration. For example, in the presence of increasing benzene, the Soret absorbance band shows only small shifts to longer wavelengths since the Soret in the presence of benzene is a combination of the TPPS peak at 413 nm and the TPPS-benzene peak at 419 nm. The difference spectrum of these two spectra clearly reveals the loss of absorbance at 413 and the increase at 419 nm due to benzene complexation of only some of the TPPS present. The combination of these 2 absorbance bands results in the small wavelength shift. Note that while naphthalene causes a change at 426 nm, benzene causes a change at 419 nm; different analytes cause different spectral changes at particular wavelengths.

The magnitude of the wavelength shift will not alter the sensitivity of the difference spectrum. The closer the wavelengths of the porphyrin and porphyrin-analyte complex, the sharper the “1st derivative” appearance of the difference spectrum and the more the change in absorbance analyte concentration. The farther apart the wavelengths, the broader the peak and trough of the difference spectrum. In all cases, the integrated area under the curve will always be proportional to analyte concentration.

It is important to note that the use of difference spectra to detect an analyte-indicator complex also means that the sensor and active surface do not need to be calibrated prior to use and that a partially-used sensor is still completely useful. This is so because, first, it is the analyte-dependent change in absorbance and not the absolute amount of indicator that is monitored. The loss of TPPS or other indicator, for example, can be quantitated from its extinction coefficient, the absorbance loss being proportional to the amount of porphyrin which reacts with analyte.

Second, since difference (comparison) spectra are used, a spectrum recorded at any previous time can be subtracted from the current spectrum to yield a measure of the change since the earlier reading. For example, the spectrum of the film at some arbitrary zero time could be subtracted from the spectrum five minutes later after exposure to analyte X. To determine if more analyte is present at, say, ten minutes, either the zero or five minute spectra could be subtracted. In the first case (i.e., t=0 subtracted) the total exposure after 10 minutes is measured; in the second case, only the change between t=5 and t=10 minutes is recorded. If in the period between 5 and 10 minutes analyte Y binds which causes an absorbance change at a different wavelength than analyte X, the difference between the zero and ten minute spectra would show a deep trough at the TPPS peak at 413 nm and 2 peaks at the wavelengths corresponding to the TPPS-X and TPPS-Y complexes. The 10 minute minus 5 minute spectrum indicates the amount of TPPS that reacted in the 5 minute period (loss in 413 nm) and the absorbance increase at the λₘₐₓ of the TPPS-Y component without interference by the TPPS-X complex formed previously. In this case, the same film can report multiple analytes. Also, the need to “calibrate” or use a fresh indicator is unnecessary since changes in the absorbance are measured regardless of the original intensity of the indicator.

More generally, it is contemplated by the instant inventor that any number of mathematical comparisons between the spectra and two different time periods could be used to accentuate the change occasioned by the introductions of low concentrations of the target compound. For example, in some cases taking the ratio of corresponding spectral intensities might be useful. In other cases, ratios (or differences, products, etc.) between the squared, cubed, etc., spectral intensities might prove effective. Obviously, many more combinations might be devised by one of ordinary skill in the art. Thus, for purposes of the instant disclosure, the term “difference” should be interpreted broadly to include any mathematical combination of a spectra intensities at one period of time with corresponding intensities at another period.

### Interaction Time

The spectral changes observed in porphyrins are typically completed within approximately 1 second in both soluble (free, aqueous) and immobilized porphyrins. Thus, for detection purposes, spectral measurements might be collected at least this often.

### Response to Changing Analyte Levels

Unless it becomes “saturated”, any detector will respond to increasing analyte levels. But, it is much less common to find a detector that can respond to decreasing levels of a compound. However, the instant disclosure provides embodiments of such a detector.

When embodiments of the instant disclosure are used in practice, the spectral measurements might come from either absorbance, fluorescence, or reflectance spectra. Where absorbance spectra are used, the preferred range of light within which measurements will be taken is the 200-900 nm (UV-VIS) range (suitable at least for chemical warfare agents). Further, it is preferable that either a dual wavelength or dual beam instrument (and procedures) be used. In the one embodiment, dual beam spectroscopy is performed using a Cary 4E UV-VIS instrument.

When dual wavelength spectroscopy is to be used, a conventional dual wavelength spectrophotometer would be a suitable instrument. Dual wavelength spectroscopy is ideally suited to measuring absorbance spectra of highly scattering turbid samples and for spectroscopic measurement via fiber optics. Since light is scattered by turbid samples, the detector tends to see the “scatter” as “absorbance” (decrease in light intensity). Further, since shorter wavelengths of light are scattered more than are longer wavelengths, the spectrum of a turbid sample in a dual beam instrument using water or air as reference has a non-flat baseline, making data interpretation difficult if not impossible.

In dual wavelength spectroscopy, two wavelengths of light alternately illuminate the sample, one wavelength being designated as a reference wavelength. Thus, a reference material need not be used. Of course, a reference wavelength should be chosen so as to not coincide with an absorbance peak of the sample. The absorbance of the reference wavelength is the “reference” signal of the system and includes light losses due to the material of the samples as well as the optical system (including cuvettes, holders, optical fibers, etc). Dual wavelength spectroscopy is the technology of choice when it is necessary to measure reflectance spectra, evanescent wave spectra, fiber optically-coupled samples, or solid/suspension/films/slurries, etc. (i.e., high scatter or variable samples, e.g., stirred).

Fluorescence spectroscopy is also suitable for use with some embodiments of the instant disclosure. Of course, it is based on different principles than absorbance spectroscopy. As is well known to those of ordinary skill in the art, in fluorescence spectroscopy a photon of light is absorbed and then emitted, the emitted photon having a longer wavelength than the photon absorbed. The time interval between absorbance and emission differentiates fluorescence from phosphorescence. The emitted photon is of lower energy (longer wavelength), the wavelength of emitted light being dictated by the energy levels of the electrons of the material.

### Spectral Deconvolution of Multiple Peaks

The presence of more than one analyte (a mixture) can be detected via the instant methods since it is quite unlikely that the analytes will have the same spectral signature. For example, if the sample is a mixture of naphthalene and benzene, a loss would be seen at 413 and a gain at 426 nm due to naphthalene and a loss at 413 and a gain at 419 due to benzene. If the monitoring instrument can optically/spectral resolve these 419 and 426 nm peaks, a trough would be seen at 413 nm and peaks at 419 and 426 nm. If the peaks cannot be resolved, a peak and a “shoulder” may be seen (which analyte is the peak and which is the shoulder depends on their relative concentrations). However, both the λₘₐₓ and absorbance of each peak can be determined accurately by calculating the 2nd derivative of the spectra. The wavelengths of the peaks will show up as troughs and the depth of the trough is proportional to absorbance which is proportional to concentration.

Spectra having multiple peaks can be manipulated using software such as Grams/32 (Galactic Industries) for subtractions, smoothing, etc. Spectra, including the 2nd and 4th derivatives or other mathematical manipulations thereof, can be manipulated using any number of available software products to determine the wavelengths of peaks and troughs and the integrated area under each curve.

### EXAMPLE APPARATUS

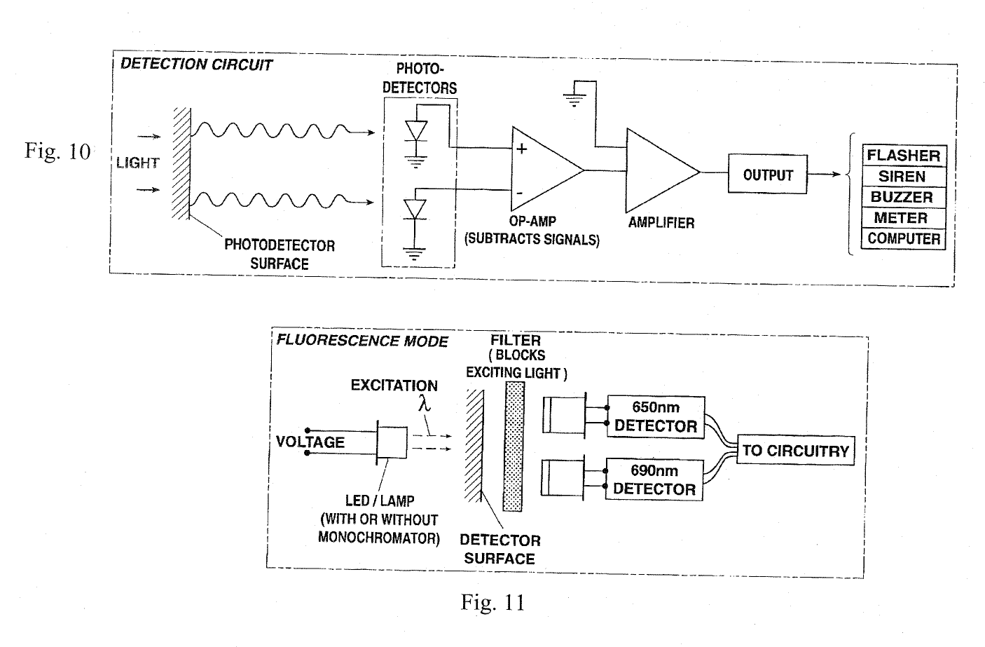

Although the instant invention might be embodied in many forms: FIGS. 10 and 11 contain some exemplary arrangements. The optical detector of the instant invention consists of several elements. First, there is a light source that can generate a single wavelength of light (e.g., a laser) or more than one wavelength of light (e.g., an LED or lamp with/without filters) that illuminates a detector surface of the type described previously. The light may be shown directly onto the surface or transmitted there via some media such as optical fibers. Further, the wavelength of the incident light can be varied through different wavelengths and/or “scanned” across the material so that different wavelengths of light are sequentially striking the material.

The detector material absorbs different amounts of the wavelengths of light falling on it, which produces an absorbance spectrum. The light not absorbed by the material is transmitted to a detector for measurement. Again, the light reflected by the detector material can be directly sensed by a detector or transmitted to a remote detector.

The change in light absorbance by the detector surface material can be measured via a conventional spectrophotometer, where the incident light is scanned through many wavelengths and the amount of light is measured at each of these frequencies to yield a spectrum. The scanning spectrophotometers can be large bench-based units, “cards” that fit into PC's and connect to the detector surface material via fiber optics, or via laptop and even smaller computers (such as those made by Ocean Optics).

Alternatively, the absorbance at one or two (or, preferably only a few) wavelengths of light can be used to detect the presence of one or a few analytes. For example, the presence of napthalene causes the porphyrin TPPS to lose its absorbance at 413 nm and to gain absorbance instead at 426 nm. In the presence of benzene, the absorbance increases at 419 nm; different analytes absorb at different wavelengths. To determine the amount of napthalene present, only the absorbance decrease at 413 nm and increase at 426 nm need be measured.

FIG. 10 diagrams one possible arrangement of a detector. The light source should be capable of at least emitting light in the general wavelength range of 400 nm-440 nm. In the preferred embodiment, the light source will be an LED or other broad band light source, but it is certainly possible that the monochromatic lights sources such as lasers could be used instead. What is important, of course, is that the light source irradiate the photodetection surface with wave lengths of light that at least interact with the specific bands of interest. Continuing with the illustration of the begun previously, the light source should include both 413 nm and 426 nm light.

As is illustrated in FIG. 10, the light will strike two photodiodes/phototransistors/photodetectors that are preferably fitted with narrow filters to admit only light at the specific frequencies of interest, i.e., 413±3 nm and 426±3-5 nm, respectively, for purposes of the instant example. The colorimetric detector material surface whose wavelength changes upon reaction with the desired analyte is preferably placed between the LED and the detectors. Alternatively, the LED light can be reflected (bounced off) the material onto the detectors (i.e., a reflectance spectra will be obtained; the same wavelengths of light will be absorbed as in the “head on” configuration.)

The output current or voltage—one can be converted to the other—of the detector is proportional to the intensity of the light striking it. Thus, in the present example, the output of the “413” detector decreases and the output of the “426” detector increase as naphthalene binds to TPPS.

A simple subtraction circuit using, for example, an operational amplifier (or “OP amp”, FIG. 10) is a preferred apparatus for analyzing the voltage difference between the two signals; the subtraction of a negative voltage (“413”) from a positive voltage (“426”) resulting in an effective addition of the two voltage current changes. This arrangement also helps prevents “false” readings. The circuitry can be devised to read or detect only when the output of the one detector goes down and the other goes up. This is a “coincidence” circuit, as both events must occur for the change to be registered. The loss of absorbance because of an increase in light or the gain in absorbance due to clouding or mud, etc. is not recorded. For purposes of the instant disclosure, the term “comparator” will be taken to include not only special purpose hardware for comparing signals (such as the differencing units utilized in the preferred embodiment), but also will be taken to encompass hardware/software combinations that allow for more elaborate comparison schemes than differences (e.g., ratios of signals, general linear combinations of signals, products, etc.), whether the operation is performed on an analog or a digital signal.

The output from the optical detectors is conventionally a voltage; with the voltage being proportional to intensity of light in the wavelength monitored which, in turn, is proportional to the amount of analyte that interacts with the detector surface.

In the case of a conformational detection system, the loss of the 413 nm peak of TPPS and an increase at some other wavelength(s) will be recorded as a nerve agent encounters the photodetector surface. Use of a proper filter on the detector allows only the wavelengths of interest to be measured. If a filter is used that passes light from, say, 420 to 430 nm, the wavelengths changes initiated by multiple analyte interactions can be recorded. Unlike the case of measuring napthalene vs. benzene using TPPS where the wavelengths are specific, it is also possible to measure a change of TPPS absorbance (or fluorescence) as the enzyme conformation changes at whatever wavelength; the specificity is not in the particular wavelength measured. The specificity is introduced into the instant system through the fact that the enzyme that will only bind specific inhibitors or substrates.

FIG. 11 is a diagram of a fluorescence-based detector where the TPPS absorbs light and emits light at 650 nm, for example. When the TPPS is altered, the fluorescence wavelengths are also altered to 690 nm in one example. Thus, the intensity of the 650 in fluorescence and the fluorescence intensity at 690 nm increases. The circuitry is the same and the additive changes are similar. In the preferred embodiment, the 650 nm and 690 nm detectors are created by placing narrow band optical filters of corresponding wavelengths ahead of broader band photodetectors.

For purposes of the instant invention a colorimetric molecule, indicator, or agent should be interpreted in its broadest sense to include a chemical compound which changes its color, absorbance spectrum, fluorescence spectrum, reflectance spectrum, and/or its fluorescence and polarization properties upon binding of or interaction with another molecule or atom. This term also encompasses those molecules whose spectral properties change upon chemical oxidation or reduction. For purposes of this disclosure, the colorimetric “indicator” can be a colorimetric compound/molecule incorporated into another molecule such as protein, DNA, RNA, nucleic acid, amino acid, peptide, etc.

It should further be noted that, although the previous discussion has principally been concerned with the real-time differencing of spectral intensities using special purpose signal processing hardware, the instant invention would work in exactly the same fashion if the differencing were performed digitally. More specifically, an analog-to-digital conversion of the detected spectral intensity signals can be performed as the information is collected, with the digital output being sent to a microprocessor or a general purpose computer (collectively a “microprocessor”, hereinafter) for subsequent digital manipulation. Of course, one advantage of this arrangement is that any mathematical operation—not just differencing—could be used to combine the information from the most recently collected spectral values with those collected earlier.

Additionally, in the preferred embodiment the light source will contain a plurality of light frequencies therein. Of course, those skilled in the art will recognize that, rather than using a single broad-band light source, instead two (or more depending on the application) narrower sources could be used instead.

As described, the instant invention teaches a new approach to the detection of toxins, viruses, bacteria, and biological factors. The invention can be configured to detect multiple target analytes in liquid or vapor (air) and operates generally by monitoring the optical properties of the receptor-indicator hybrid that is altered by interaction with the target analyte/agent. In more particular, target analytes can be detected by monitoring changes in the optical properties of the absorbance, reflectance, and/or fluorescence spectra of highly colored heterocyclic compounds such as porphyrins or related compounds such as phthalocyanines. Further, the instant apparatus and method is unique in that it does not require pre-use “calibration” of the colorimetric materials or apparatus. Because the instant invention continuously monitors the detecting surface/volume and compares its spectrographic properties with those of the detecting surface a few moments before, the methods and devices disclosed herein are, to a large extent, self-calibrating.

While the invention has been described with a certain degree of particularity, it is understood that the invention is not limited to the embodiment(s) set forth herein for purposes of exemplification, but is to the limited only by the scope of the attached claim or claims, including the full range of equivalency to which each element thereof is entitled.

What is claimed is:

1. A method of detecting a biological entity comprising:
covalently bonding a colorimetric indicator to a receptor capable of binding said biological entity to create a receptor-indicator hybrid, the colorimetric indicator having a known spectrographic profile;
binding the receptor-colorimetric hybrid to a testing substrate;
exposing the testing substrate in a test environment;
performing a spectral interrogation of the receptor-indicator hybrid to determine a change in the spectrographic profile thereof; and
determining the binding of a specified biological based upon the change the spectrographic profile.

2. The method of claim 1, wherein the steps of exposing the testing substrate, performing a spectral interrogation, and determining the binding of a specified analyte occur in real time.

3. The method of claim 1, wherein the spectral interrogation includes the measurement of at least two spectral values.

4. The method of claim 3, wherein the step of determining the binding of a specified analyte based upon the change in the spectra is based upon a change in absorption of at least one predetermined wavelength.

5. The method of claim 1, wherein the colorimetric indicator is a porphyrin.

6. The method of claim 1, wherein the receptor-indicator hybrid binds to at least a portion of a toxin.

7. The method of claim 1, wherein the receptor-indicator hybrid binds to at least a portion of a virus.

8. The method of claim 1, wherein the receptor-indicator hybrid binds to a component of a virus.

9. The method of claim 1, wherein the receptor-indicator hybrid binds to at least a portion of a bacterium.

10. The method of claim 1, wherein the receptor-indicator hybrid binds to a component of a bacterium

11. The method of claim 1, wherein the spectral interrogation is by evanescent light spectroscopy.

12. The method of claim 1, wherein the spectral interrogation is by fluorescence spectroscopy.

13. The method of claim 1, wherein the porphyrin is bound at the site of the receptor where the analyte will directly bind.

14. The method of claim 1, wherein the porphyrin is bound at the site of the receptor other than where the analyte will directly bind but such that the binding alters the spectral properties of the colorimetric indicator.

15. The method of claim 1, wherein the biological remains bound to the immobilized receptor and is available for further examination or investigation.

16. The method of claim 1, wherein the receptor molecule is selected from the group consisting of a monomer, a polymer, or macromolecule of amino acids, a nucleic acids, carbohydrates/saccharides, or lipids.

17. The method of claim 1, wherein the colorimetric indicator exhibits light absorbance.

18. The method of claim 1, wherein the colorimetric indicator exhibits light fluorescence/phosphorescence.

19. The method of claim 1, where the colorimetric indicator is selected from the group consisting of a porphyrin and a phthalocyanine.

20. The method of claim 1, wherein the colorimetric indicator is selected from the group consisting of a colored compound and a colored reagent.
